(19) United States
(12) Patent Application Publication
(10) Pub. No.: US 2025/0049980 A1
(43) Pub. Date: Feb. 13, 2025

(54) IONIZATION UNIT

(71) Applicant: NITYA INNOVATIONS LLP, Panvel (IN)

(72) Inventor: NILESH PUNDLIK GAWALI, Panvel (IN)

(73) Assignee: NITYA INNOVATIONS LLP, Panvel, MH (IN)

(21) Appl. No.: 18/720,659

(22) PCT Filed: Dec. 10, 2022
(86) PCT No.: PCT/IN2022/051065

§ 371 Date: Jun. 17, 2024

(30) Foreign Application Priority Data
Dec. 16, 2021 (IN) 202121058749

(51) Int. Cl. A61L 9/22

(52) U.S. Cl. CPC A61L 9/22; A61L 2209/14

(57) ABSTRACT

The present invention discloses an ionization unit configured to produce one or more types of Reactive Oxygen Species (ROS). The ionization unit includes an inlet port, first electrode, a second electrode, one or more high voltage generators and an outlet port. The inlet port facilitates entry of a predefined gaseous composition into the ionization unit. The first electrode and second electrode are maintained at a first and second set of ionization parameters respectively. The high voltage generators are operationally coupled to the first electrode and the second electrode to enable the electrodes to generate Corona discharge. The outlet port produces a continuous stream of ROS. The first set of ionization parameters is different from the second set of ionization parameters.

### FIELD OF INVENTION

The present invention relates to an ionization unit. More specifically, the present invention relates to a disinfecting ionization unit.

### BACKGROUND

Disinfection (or sterilization/purification) is a safety practice generally observed in many public and private settings to maintain and improve hygienic conditions of one's surroundings. The term is commonly used to define methods to tackle problems associated with the presence of microbial population (bacteria, fungi, viruses, etc.) and other suspended particles (foul odor, atmospheric pollutants, etc.). One can disinfect an environment with many available options ranging from conventional mopping of surfaces with disinfectant solutions to providing sophisticated air purification systems.

However, none of the conventional solutions provide an “all-in-one” method of disinfection, i.e., surface disinfection as well as air disinfection. Even the latest method of disinfection via UV treatment fails to properly penetrate many surfaces resulting in lack of disinfection where shadows can form (with respect to the UV source). Hence, UV treatment is only efficient in open spaces having clear line of sight with a UV source.

Fumigation is the go-to approach for a comprehensive disinfection of a defined closed space. But the process itself requires strict supervision and is very cumbersome to carry out. Further, fumigation often leaves lingering fumes which are outright toxic to human health.

Many have tried to implement disinfection by circulating ionized gases which has proven to be a promising alternative. But the process is heavily dependent on ambient temperature and humidity levels, which varies seasonally and so does the efficiency of said process. To circumvent the aforesaid, a hydrogen peroxide cartridge is often used which leads to a non-functioning system when the cartridge is spent. Moreover, the conventional systems, due to rudimentary electrodes, produce an undesirable high pitch sound while generating Corona discharge for ionization. Further, the corona discharges are inconsistent resulting in unpredictable ionization.

Therefore, there arises a requirement for an ionization unit that can overcome the aforementioned problems.

### SUMMARY

The present invention relates to an ionization unit configured to produce one or more types of Reactive Oxygen Species (ROS). The ionization unit includes an inlet port, first electrode, a second electrode, one or more high voltage generators and an outlet port. The inlet port facilitates entry of a predefined gaseous composition into the ionization unit. The first electrode is maintained at a first set of ionization parameters selected from one or more predefined ionization parameters. The second electrode is maintained at a second set of ionization parameters selected from one or more predefined ionization parameters. The high voltage generators are operationally coupled to the first electrode and the second electrode to enable the electrodes to generate Corona discharge. The outlet port produces a continuous stream of ROS. The first set of ionization parameters is different from the second set of ionization parameters. The first electrode interacts with the predefined gaseous composition flowing from the inlet port to produce an intermediary gaseous composition. The second electrode interacts with the intermediary gaseous composition to produce ROS. The inlet port, the first electrode, the second electrode and the outlet port define a fluid flow path of the ionization unit.

The foregoing features and other features as well as the advantages of the invention will become more apparent from the following detailed description, which proceeds with reference to the accompanying figures.

### BRIEF DESCRIPTION OF DRAWINGS

The summary above, as well as the following detailed description of illustrative

embodiments, is better understood when read in conjunction with the apportioned drawings. For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale.

FIG. 1 depicts an ionization unit 100 in accordance with an embodiment of the present invention.

FIG. 2 depicts a first electrode 120a in accordance with an embodiment of the present invention.

FIG. 3 depicts an ionization unit 100 in accordance with an embodiment of the present invention.

FIG. 4a-c depicts different configuration of the ionization unit 100 in accordance with an embodiment of the present invention.

FIG. 5 depicts a method 500 to operate the ionization unit 100 in accordance with an embodiment of the present invention.

### DETAILED DESCRIPTION OF THE DRAWINGS

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like; Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Although the operations of exemplary embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show the various ways in which the disclosed system, method, and apparatus can be used in combination with other systems, methods, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and apportioned claims, or may be learned by the practice of embodiments as set forth hereinafter.

In accordance with the present disclosure, an ionization unit configured to produce one or more types of Reactive Oxygen Species (ROS) and method of ionization thereof is disclosed. The ionization unit helps to selectively neutralize bacterial, fungal and viral population in a predefined area using ROS. The predefined area may be an enclosed space with a ceiling. The ionization unit may automatically adjust the amount of ROS produced per unit time based on the predefined area.

The ionization unit further helps to remove undesirable suspended particulate matter and foul odors from the vicinity. The ionization unit is not fatal to organisms other than microbes and does not adulterate the surroundings.

The ionization unit of the present invention is a low maintenance solution to be implemented in any setting (industrial, commercial, residential, etc.). The ionization unit does not require any special condition (temperature, humidity, etc.) to function properly. In other words, the ionization unit can be operated at full efficiency irrespective of current humidity and temperature associated with seasonal changes.

The ionization unit of the present invention includes two electrodes maintained at different parameters for example, temperature, pressure and electric field, producing consistent rotating Corona discharge for ionization. The said arrangement enables operation of the ionization unit independent of any refillable/cartridge (like a water vapor source) system thereby providing efficient operation without any downtime.

The ionization unit of the present invention may include one or more configurable auto-scheduler which automatically supervises the runtime of the ionization unit. Additionally/alternatively, the user may manually control the ionization unit via a physical interface. The physical interface may include a remote, an LCD/LED screen with physical buttons, a touch screen or a combination thereof.

Further, the ionization unit of the present invention can be configured to operate in definite disinfection cycles where each cycle may extend for a few minutes in a configurable manner without sacrificing efficiency of the ionization unit. In an exemplary embodiment, the ionization unit of the present invention is configured to perform eight disinfection cycles (each cycle lasting up to 15-140 minutes depending on the size of the predefined area) every 24 hours thereby saving power. However, the ionization unit of the present invention may be operated for long hours at the discretion of the end user.

The ionization unit may be remotely controlled via Bluetooth, WiFi, infrared or other data transmission technology. The ionization unit may also be controlled via mobile devices (smartphones, laptop, home automation) using Web APIs. The runtime logs and maintenance logs of the ionization unit may be saved in a cloud storage for easy retrieval and subsequent analysis. In an exemplary embodiment, an ion sensor (senses ambient ionic concentration of a predefined ionic species) operationally coupled to the ionization unit, controls the one or more modes of operation along with Ionization parameters to maintain a predefined ionic concentration in an enclosed space.

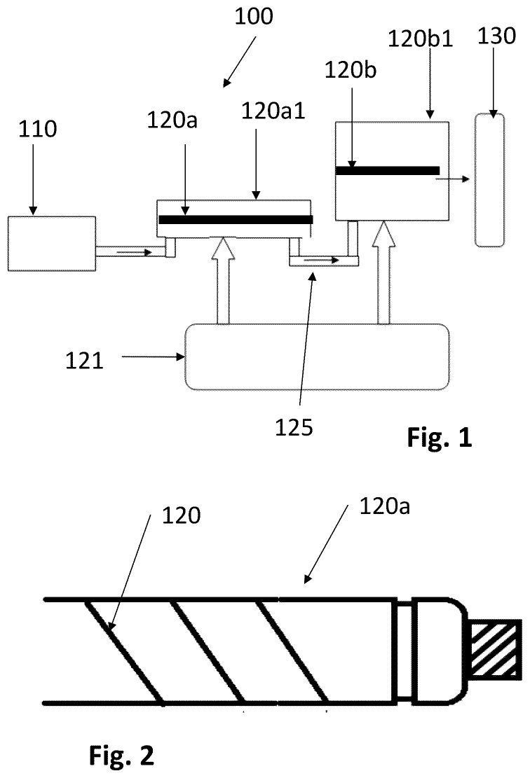

FIG. 1 depicts an ionization unit 100 of the present invention. The ionization unit 100 includes a plurality of components including but not limited to an inlet port 110, two or more electrodes, high voltage generator 121 and an outlet port 130. In an exemplary embodiment, two or more electrodes include a first electrode 120a and a second electrode 120b. The ionization unit 100 also includes a fluid flow path defined by the directional flow of a fluid (for example, atmospheric air) within the ionization unit 100. In an exemplary embodiment, the fluid flow path originates at the inlet port 110; continues through the two or more electrodes 120a, 120b placed sequentially one after the other and ends at the outlet port 130.

Additionally/optionally, the ionization unit 100 may be disposed inside a closed housing (not shown) for easy deployment as required. The housing may offer portability to the ionization unit 100.

The inlet port 110 of the ionization unit 100 may act as an entrance to the fluid flow path. In an exemplary embodiment, the inlet port 110 is a porous surface area provided on the housing of the ionization unit 100. The inlet port 110 of the ionization unit 100 may facilitate entry of a predefined gaseous composition into the ionization unit 100. In an embodiment, the predefined gaseous composition includes atmospheric air.

The inlet port 110 may include a passive mode or an active mode. In the passive mode, the inlet port 110 relies on natural gaseous movement to facilitate entry of the predefined gaseous composition into the ionization unit 100.

In the active mode, the inlet port 110 relies on a blowing means to facilitate entry of the predefined gaseous composition into the ionization unit 100. In an embodiment, the blowing means includes an air pump. The blowing means collects the predefined gaseous composition and forces it into the fluid flow path of the ionization unit 100. The blowing means may have an adjustable RPM (revolution per minute) corresponding to the pressure generated inside the ionization unit 100. The adjustable RPM, without limitation, may define the operation parameter of the ionization unit 100.

The inlet port 110 may be provided with a filtering means to restrict entry of dust particles into the ionization unit 100. In an exemplary embodiment, a simple fibrous filter is coupled to the inlet port 110 to prevent entry of fine dust particles into the ionization unit 100.

As depicted in FIG. 1, the first electrode 120a and the second electrode 120b are arranged

one after the other within the fluid flow path. The first electrode 120a and the second electrode 120b may each include one or more electrodes. The predefined gaseous composition coming from the inlet port 110 interacts with the first electrode 120a and then the second electrode 120b. In an embodiment, the one or more electrodes 120a, 120b are made of stainless-steel machined cylinder with tolerance of ±0.25 mm. In an exemplary embodiment, the first electrode 120a includes a diameter of 18 mm and the second electrode 120b includes a diameter of 3 mm.

Although the ionization unit 100 of the present invention is described with the first electrode 120a and the second electrode 120b; the teachings of the present application can be easily applied to include a third electrode, a fourth electrode and so on.

The first electrode 120a and the second electrode 120b of the ionization unit 100 may be maintained at different parameters for example, a predefined temperature difference, pressure and electric field (described below in detail). The said properties, without limitation, of the one or more electrodes 120a, 120b at any given time may define the ionization parameters of the ionization unit 100. In other words, the first electrode 120a may be maintained at a first set of ionization parameters and the second electrode 120b may be maintained at a second set of ionization parameters selected from the predefined ionization parameters. In an exemplary embodiment, the first set of ionization parameters are different from the second set of ionization parameters.

The first electrode 120a and the second electrode 120b may be operationally coupled to one or more high voltage generators 121. The high voltage generators 121 enables the first and second electrodes 120a, 120b to generate rotating Corona discharge (or Corona discharge). The rotating Corona discharge may be pre-modulated via the high voltage generators 121 to produce a pleasant sound. The sound also enables a user to ensure proper functioning of the ionization unit 100.

As depicted in FIG. 2, at least one of the first and second electrodes 120a, 120b say a first electrode 120a, may include a spirally engraved path (or grooves) 120 on the first electrode 120a. In an exemplary embodiment, the grooves 120 include a pitch of 9 mm. Other functionally equivalent structure is within the scope of the teachings of the present invention. In an exemplary embodiment, the first electrode 120a is maintained at reference ground potential.

The first electrode 120a may be enclosed inside a first container 120a1 such that the container becomes an integral part of the fluid flow pathway. The first container 120a1 may be made of an inert material. In an exemplary embodiment, the first container 120a1 is made of high temperature glass such as borosilicate. A gap between the first electrode 120a and the first container 120a1 maintains a flow rate of the predefined gaseous composition as received from the inlet port 110.

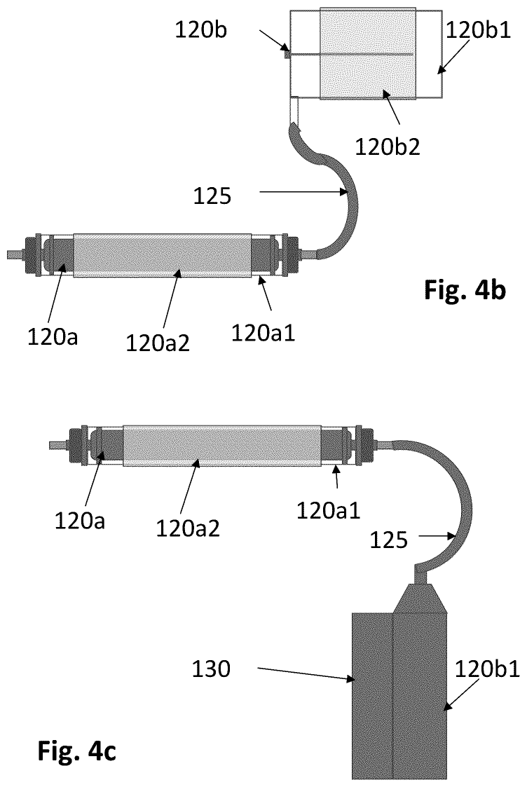

Additionally and/or optionally, as depicted in FIG. 4a, the first electrode 120a may include a coil structure 120a2 of one or more wires wound around the first container 120a1. The one or more wires may be made of a material including a metal or a metallic alloy. The coil structure 120a2 defines the terminals for the first electrode 120a, i.e., the high voltage generators 121 may be electrically coupled to the first electrode 120a via the coil structure 120a2.

During operation of the ionization unit 100, a magnetic field is created along the axis of the first electrodes 120a due to the flow of current through the coil structure 120a2 wound over the first container (dielectric) 120a1. In the presence of the said magnetic field, the groove 120 enables the rotating Corona discharge to move along the axis of the first electrode 120a. The grooves 120 also help in generating consistent rotating Corona discharge from the first electrode 120a.

The rotating Corona discharge partially or completely ionizes the predefined gaseous composition present within the immediate surrounding of the first and second electrodes 120a, 120b thereby producing reactive oxygen species (ROS). Scientifically, ROS includes superoxide anion (O²⁻), hydrogen peroxide (H₂O₂), hydroxyl radical (HO^{·}), etc. The said ROS helps to selectively neutralize bacterial, fungal and viral population in a predefined area. Further, the said ROS are attracted to the suspended particulate matter and forces them to settle down thereby purifying the air.

The first electrode 120a interacts with the predefined gaseous composition flowing from the inlet port 110. The first container 120a1 may include at least two openings for enabling the flow of the predefined gaseous composition around the first electrode 120a. In an exemplary embodiment, the inlet port 110 is operationally coupled to one of the openings of the first container 120a1.

The first electrode 120a may include a means to facilitate the predefined gaseous composition from the inlet port 110 to pass through the first electrode 120a, which helps to increase the temperature of the predefined gaseous composition. The rotating Corona discharge from the first electrode 120a may increase the temperature of the first electrode 120a. The change in temperature of the first electrode 120a may range from 15 degrees to 25 degrees depending on the electric field provided by the high voltage generators 121 (and/or the corresponding mode of operation of the ionization unit 100). Further, due to the active mode of the inlet port 110, pressure around the first electrode 120a increases compared to the ambient atmospheric pressure. The predefined gaseous composition after interacting with the first electrode 120a produces an intermediary gaseous composition. In an exemplary embodiment, the intermediary gaseous composition includes at least one of the following entities O⁻, OH⁻, NO, H₂O₂, O₃⁻, H⁺, etc.

Similar to the first electrode 120a, the second electrode 120b may be enclosed inside a second container 120b1. The second container 120b1 may be made of an inert material including but not limited to ABS (Acrylonitrile Butadiene Styrene) plastic, borosilicate glass, etc. In an exemplary embodiment, the second container 120b1 is made of borosilicate glass. The second container 120b1 may be larger than the first container 120a1. A diameter of the second container 120b1 may at least be double of a diameter of the first container 120a1. In an exemplary embodiment, the diameter of the second container 120b1 is 36 mm.

The second container 120b1 may include at least two openings for enabling the flow of the intermediary gaseous composition around the second electrode 120b. The second container 120b1 may be operationally coupled to the first container 120a1 via a tube 125. In an exemplary embodiment, the tube 125 connects the fluid pathway between the first electrode 120a and the second electrode 120b by making fluid contact between at least one opening of each of the first container 120a1 and the second container 120b1. The tube 125 may be made of a material including but not limited to flexible silicon rubber, high temperature glass, ABS, etc. The tube 125 may have a very thin profile to provide a compressing effect on the intermediary gaseous composition. In an exemplary embodiment, the tube 125 have a diameter of 6 mm.

Additionally and/or optionally, as depicted in FIG. 4a, the second container 120b1, when made up of borosilicate glass or other fragile material, may include a layer of a protective material 120b2. In an exemplary embodiment, the protective material 120b2 includes polystyrene foam. The protective material 120b2 acts as cushion while the second container 120b1 is mounted inside a holding bracket/stand.

Thus, the intermediary gaseous composition enters the second container 120b1 from the first container 120a1 via the tube 125 and expands. The sudden expansion of the intermediary gaseous composition within the second container 120b1 rapidly reduces the pressure and temperature around the second electrode 120b. This further leads to condensation of the vapours (preferably water vapours) present in the intermediary gaseous composition over the second electrode 120b as a plurality of micro droplets. The plurality of micro droplets may also remain in a suspended form within the second container 120b1. The ability of the ionization unit 100 of the present invention to form the plurality of micro droplets around the second electrode 120b, makes the operation of the ionization unit 100 independent of seasonal variation of humidity levels in the atmospheric air.

The plurality of micro droplets may then be exposed to a modulated high voltage electric field up to 5 kV from the second electrode 120b in the presence (bombardment) of other ionic species of the intermediary gaseous composition coming from the first container 120a1 to produce ROS. In an exemplary embodiment, the plurality of condensed micro droplets are ionised to form hydroxyl ions.

In an exemplary embodiment, the coil structure 120a2 of the first electrode 120a is maintained at a nominal high voltage in the range of 10 kV. The said high voltage is regulated by a controlling means (further described below) in real time. The energy consumed by the first electrode 120a for ionization is proportional to the high voltage electric field generated and controlled by the high voltage generator 121. The said energy further influences temperature difference (say a temperature delta) of the passing gases over the one or more electrodes 120a, 120b. The said temperature delta governs the condensation of vapours (say, a condensation rate) and hence the concentration of the ROS generated by the second electrode 120b.

Further, the degree of ionization (or ion concentration) may be directly proportional to an intensity of applied electric field from the high voltage generators 121 to the first and second electrodes 120a, 120b. The required electric field is controlled from high voltage generator 121 depending on the operation mode configured to generate concentration of ions by the ionization unit 100.

The ionization unit 100 may include one or more predefined modes of operation. In an exemplary embodiment, the ionization unit 100 is provided with two modes of operation, a normal mode and a mild mode corresponding to different degree of ionization. The normal mode may generate relatively more ROS than the mild mode. In the absence of any humans and/or pets, the ionization unit 100 may be operated in the normal mode. In the presence of any humans and/or pets, the ionization unit 100 may be operated in the mild mode. The user may selectively operate the ionization unit 100 in one of the one or more predefined modes of operation at any given time. The normal mode and the mild mode may correspond to different temperature deltas (as described above).

The outlet port 130 may be operationally coupled to the second container 120b1, thereby ending the fluid flow path of the ionization unit 100. In an exemplary embodiment, the outlet port 130 is a porous area provided on the housing of the ionization unit 100.

Similar to the inlet port 110, the outlet port 130 may include an active mode and a passive mode. In the active mode, the outlet port 130 may help in evaporation of the plurality of micro droplets thereby further reducing the temperature of the second electrode 120b.

The outlet port 130 produces a continuous stream of ROS generated by the second electrode 120b. In an exemplary embodiment, ROS generated at a flow rate of 4-6 lpm by the second electrode 120b is mixed with a typical flow of 800-900 lpm generated by the outlet port 130, thereby facilitating an excellent ionization efficiency required for sustainable performance of the ionization unit 100.

In an embodiment, the outlet port 130 may be operationally coupled to an existing air circulation system to efficiently distribute the ROS around the predefined area.

In general, the flow within the fluid flow pathway from the inlet port 110 to the outlet port 130 may also have a cooling effect on the one or more electrodes 120a/120b and the high voltage generator 121.

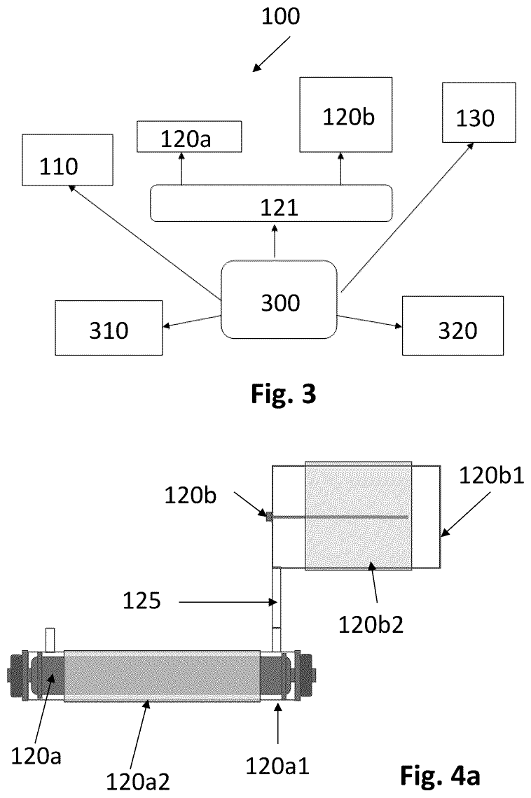

As depicted in FIG. 3, the components of the ionization unit 100 may be coupled to a controlling means 300. The controlling means 300 may include without limitation a microcontroller, a current limiting circuitry for high voltage generator 121, a high voltage electric field monitoring section, an air pump on-off control section, etc. In an exemplary embodiment, as shown in FIG. 3, a microcontroller is operationally coupled to the inlet port 110, the one or more high voltage generator 121, and the outlet port 130. The controlling means 300 may influence the ionization parameters as well as the operation parameters of the ionization unit 100. The controlling means 300 may also monitor any faults during operation and help in troubleshooting the ionization unit 100. In an exemplary embodiment, the controlling means 300 is configured to provide audio visual error beeps/codes if a fault is found in the ionization unit 100.

In an embodiment, the controlling means 300 turn on/off and/or set rpm (rotation per minute) of the inlet port 110 and the outlet port 130. The controlling means 300 also instructs the one or more HV generators 121 to turn on/off and/or set the electric field of the two or more electrodes 120a, 120b coupled to the one or more HV generators 121. Further, the electric field from the high voltage generators 121 can be modulated by the controlling means 300, at a resonating frequency and time varying amplitude depending on the selected mode of operation of the ionization unit 100.

The controlling means 300 may be provided with a user interface 310 to program, access existing programs, schedule a disinfection cycle (definite duration and/or time of operation), control, etc. the ionization unit 100 of the present invention. The user interface 310 may include at least a display and a human interface device (keypad, touchscreen, etc.). In an exemplary embodiment, the controlling means 300 auto-schedules the ionization unit 100 to operate in definite disinfection cycles where each cycle may extend for a few minutes (depending on the size of the predefined area) in a configurable manner without sacrificing efficiency of the ionization unit 100.

The controlling means 300 may also be provided with a wireless unit 320. The wireless unit 320 may include without limitation a Bluetooth module, an IoT module, etc. The wireless unit 320 may enable the user to perform the function of the user interface 310 remotely via other electronic devices. In an exemplary embodiment, a mobile device is used to control the ionization unit 100 via the wireless unit 320.

In an exemplary embodiment, the decisions of the controlling means 300 is governed by a feedback sensor monitoring ambient physiological parameters. The feedback sensor may include a wireless or a wired sensor. The ambient physiological parameters include but are not limited to temperature, pressure, humidity, concentration of ionic (ROS) species, etc.

The ionization unit 100 may be assembled in a different configuration based on the user's requirements. FIG. 4a-c depicts a few exemplary configurations in which the ionization unit 100 may be assembled. It may be noted that based on the teachings of the present invention, other assembly configurations can be envisioned by a person skilled in the art.

FIG. 4a depicts an exemplary configuration of the ionization unit 100 where the first electrode 120a is placed perpendicular to the fluid flow pathway. Further, the second container 120b1 is operationally coupled to the first container 120a1 via a high temperature glass tube 125.

FIG. 4b depicts another exemplary configuration of the ionization unit 100 where the first electrode 120a is placed inline to the fluid flow pathway. Further, the second container 120b1 is operationally coupled to the first container 120a1 via a flexible silicon rubber tube 125. The embodiment depicted in FIG. 4b is cheaper to manufacture. Further, the embodiment depicted in FIG. 4b is easily mounted in horizontal as well as vertical configuration at the site of operation.

FIG. 4c depicts yet another exemplary configuration of the ionization unit 100 where the first electrode 120a is placed inline to the fluid flow pathway. The second container 120b1 is made of ABS. The second container 120b1 is operationally coupled to the first container 120a1 via a flexible silicon rubber tube 125. And the second container 120b1 forms a single unit with the outlet port 130. Further, the second container 120b1 includes two second electrodes 120b. The embodiment depicted in FIG. 4c provides better air flow. Further, the embodiment depicted in FIG. 4c provides easy scalability in terms of ion generation capacity of the ionization unit 100.

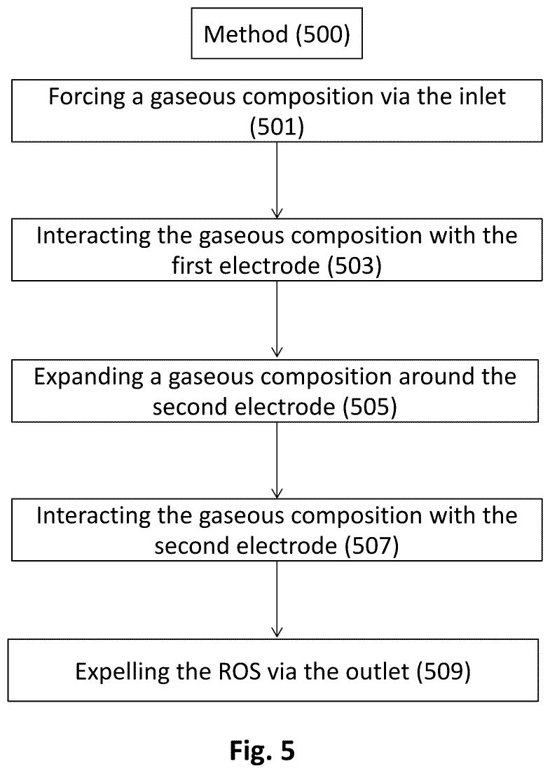

As depicted in FIG. 5, a method 500 of operation of the ionization unit 100 of the present invention includes a series of treatment steps of the predefined gaseous composition. The resulting ROS from the said method 500 helps to selectively neutralize bacterial, fungal and viral population in a predefined area. It further helps to remove undesirable suspended particulate matter and foul odors from the vicinity. In an exemplary embodiment, the predefined area ranges from 100 sq. feet to 1000 sq. feet with a ceiling height not extending 11 feet. The ionization unit 100 may be configured to operate as per the predefined area by changing the ionization parameters and/or operation parameters of the inlet port 110, the first electrode 120a, the second electrode 120b, high voltage generator 121, and the outlet port 130 as required.

The method 500 begins at step 501, which may include forcing the predefined gaseous composition into the ionization unit 100 via the inlet port 110. In an exemplary embodiment, the blowing means collects the predefined gaseous composition and forces it into the fluid flow path of the ionization unit 100 via the inlet port 110.

At the next step 503, the predefined gaseous composition may interact with the first

electrode 120a to produce the intermediary gaseous composition. The first electrode 120a is maintained at a relatively high temperature and pressure compared to the second electrode 120b which facilitates an excellent ionization efficiency. In an exemplary embodiment, the predefined gaseous composition is exposed to the rotating Corona discharge of the first electrode 120a to produce the following entities O⁻, OH⁻, NO, H₂O₂, O₃⁻, H⁺, etc.

At the next step 505, the intermediary gaseous composition may expand around the second electrode 120b which rapidly reduces the pressure and temperature around the second electrode 120b. This further leads to condensation of the vapours (preferably water vapours) present in the intermediary gaseous composition over the second electrode 120b as a plurality of micro droplets. The plurality of micro droplets may also remain in a suspended form within the second container 120b1.

At the next step 507, the intermediary gaseous composition may interact with the second electrode 120b to produce ROS. In other words, the plurality of micro droplets may get exposed to the Corona discharge from the second electrode 120b in the presence (bombardment) of other ionic species of the intermediary gaseous composition coming from the first electrode 120a to produce ROS (preferably hydroxyl ions).

At the next step 509, the ROS is expelled out of the ionization unit 100 via the outlet port 130. The expelled ROS effectively neutralizes bacterial, fungal and viral population in the predefined area and removes undesirable suspended particulate matter and foul odors from the vicinity.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

We claim:

1. An ionization unit (100) configured to produce one or more types of Reactive Oxygen Species (ROS), the ionization unit (100) comprising:
an inlet port (110) facilitating entry of a predefined gaseous composition into the ionization unit (100);
a first electrode (120a) maintained at a first set of ionization parameters selected from one or more predefined ionization parameters;
a second electrode (120b) maintained at a second set of ionization parameters selected from one or more predefined ionization parameters;
one or more high voltage generators (121) operationally coupled to the first electrode (120a) and the second electrode (120b) to enable the electrodes (120a, 120b) to generate Corona discharge; and
an outlet port (130) producing a continuous stream of ROS;
wherein, the first set of ionization parameters is different from the second set of ionization parameters;
wherein, the first electrode (120a) interacts with the predefined gaseous composition flowing from the inlet port (110) to produce an intermediary gaseous composition;
wherein, the second electrode (120b) interacts with the intermediary gaseous composition to produce ROS; and
wherein, the inlet port (110), the first electrode (120a), the second electrode (120b) and the outlet port (130) define a fluid flow path of the ionization unit (100).

2. The ionization unit (100) as claimed in claim 1, wherein the inlet port (110) and the outlet port (130) include one of a passive mode or an active mode.

3. The ionization unit (100) as claimed in claim 1, wherein the inlet port (110) is provided with a filtering means.

4. The ionization unit (100) as claimed in claim 1, wherein the one or more predefined ionization parameters include temperature difference, pressure and electric field.

5. The ionization unit (100) as claimed in claim 1, wherein the first electrode (120a) and the second electrode (120b) are enclosed inside a first container (120a1) and a second container (120b1) respectively.

6. The ionization unit (100) as claimed in claim 5, wherein the first electrode (120a) includes a coil structure (120a2) of one or more wires wound around the first container (120a1).

7. The ionization unit (100) as claimed in claim 1, wherein the first electrode (120a) includes a spirally engraved groove (120).

8. The ionization unit (100) as claimed in claim 5, wherein a diameter of the second container (120b1) is at least double of a diameter of the first container (120a1).

9. The ionization unit (100) as claimed in claim 5, wherein the second container (120b1) is operationally coupled to the first container (120a1) via a tube (125).

10. The ionization unit (100) as claimed in claim 1, wherein the ionization unit (100) includes a controlling means 300 to control the one or more predefined ionization parameters as well as one or more operation parameters of the ionization unit (100).

11. The ionization unit (100) as claimed in claim 1, wherein the one or more operation parameters include adjustable RPM of the inlet port (110) and the outlet port (130) in active mode.

12. The ionization unit (100) as claimed in claim 1, wherein the first electrode (120a) is placed perpendicular to the fluid flow pathway.

13. The ionization unit (100) as claimed in claim 1, wherein the first electrode (120a) is inline with the fluid flow pathway.

14. The ionization unit (100) as claimed in claim 5, wherein the second container (120b1) forms a single unit with the outlet port (130).

15. The ionization unit (100) as claimed in claim 1, wherein the second electrode (120b) includes two electrodes.

16. A method (500) of operation of an ionization unit (100), the method comprising:
forcing a predefined gaseous composition into the ionization unit (100) via an inlet port (110);
interacting the predefined gaseous composition with a first electrode (120a) to produce an intermediary gaseous composition;
expanding the intermediary gaseous composition around a second electrode (120b);
interacting the intermediary gaseous composition with the second electrode (120a) to produce Reactive Oxygen Species (ROS); and
expelling the ROS out of the ionization unit (100) via an outlet port (130).
